(19) United States
(12) Patent Application Publication
(10) Pub. No.: US 2024/0269204 A1
(43) Pub. Date: Aug. 15, 2024

(54) COMPOSITION FOR ALLEVIATING SKIN DAMAGE CAUSED BY HYPOXIA CONTAINING SACCHAROMYCES CEREVISIAE STRAIN

(71) Applicant: AMOREPACIFIC CORPORATION, Seoul (KR)

(72) Inventors: Eunmi KIM, Yongin-si (KR); Nok Hyun PARK, Yongin-si (KR)

(73) Assignee: AMOREPACIFIC CORPORATION, Seoul (KR)

(21) Appl. No.: 18/436,603

(22) Filed: Feb. 8, 2024

(30) Foreign Application Priority Data
Feb. 9, 2023 (KR) 10-2023-0017160
Jan. 19, 2024 (KR) 10-2024-0008677

(51) Int. Cl. A61K 36/064; A61K 8/9728; A61K 36/82; A61P 17/00; A61Q 19/08; C12N 1/18; C12R 1/865

(52) U.S. Cl. CPC A61K 36/064; A61K 8/9728; A61K 36/82; A61P 17/00; A61Q 19/08; C12N 1/185; A61K 2236/333; C12R 2001/865

(57) ABSTRACT

The present specification relates to a composition for alleviating skin damage due to hypoxia, which contains one or more selected from the group consisting of a Saccharomyces cerevisiae strain; its lysate; its culture; and an extract of the strain, lysate, or culture as an active ingredient, and a novel Saccharomyces cerevisiae strain, and the composition, which contains one or more selected from the group consisting of a Saccharomyces cerevisiae strain; its lysate; its culture; and an extract of the strain, lysate, or culture as an active ingredient, according to an aspect of the present invention is effective in inhibiting the expression of MMP1 and COX2 overexpressed in skin in a hypoxic environment, and is thus excellently effective in improving skin damaged by exposure to a hypoxic environment for a long period of time or repeatedly or alleviating skin damage due to hypoxia.

### CROSS-REFERENCE TO RELATED APPLICATION

This application claims priority to and the benefit of Korean Patent Application No. 10-2023-0017160, filed on Feb. 9, 2023, and Korean Patent Application No. 10-2024-0008677, filed on Jan. 19, 2024, the disclosures of which are incorporated herein by reference in its entirety.

### INCORPORATION BY REFERENCE OF SEQUENCE LISTING

The content of the electronically submitted sequence listing, file name: OF22P140US_sequence list.xml; size: 4.5 kilobytes; and date of creation: Jan. 22, 2024, filed herewith, is incorporated herein by reference in its entirety.

### BACKGROUND OF THE INVENTION

### Field of the Invention

The present specification relates to a composition for alleviating skin damage due to hypoxia, which contains one or more selected from the group consisting of a Saccharomyces cerevisiae strain; its lysate; its culture; and an extract of the strain, lysate, or culture as an active ingredient, and a novel Saccharomyces cerevisiae strain.

### Description of the Related Art

The skin is a part of the body that is directly exposed to the external environment, and not only acts as a protective layer to protect the body's important organs but also regulates moisture evaporation and protects the body from external infections. However, no matter how well the skin blocks viral invasion from the outside, external stress such as excessive ultraviolet rays and a polluted environment causes skin irritation and ultimately leads to skin aging.

Since the recent outbreak of coronavirus disease-19 (COVID-19), the skin has been thus exposed to hypoxia for a long period of time as COVID-19 has entered an endemic in which COVID-19 does not end quickly but spreads for a long period of time and wearing masks has become routine. Regardless of COVID-19, the skin may be exposed to a hypoxic environment during excessive exercise.

Since skin damage may be induced by such a hypoxic environment, the present inventors have studied a method to alleviate skin damage due to hypoxia, and as a result, isolated a novel Saccharomyces cerevisiae strain, verified its effect on the alleviation of skin damage due to hypoxia, and thus completed the present invention.

### SUMMARY OF THE INVENTION

The present inventors have conducted studies on ingredients that can alleviate skin damage due to hypoxia, and as a result, verified the efficacy of a Saccharomyces cerevisiae strain newly discovered from green tea flowers and its extract in improving skin damage due to hypoxia.

Accordingly, in an aspect, an object of the present invention is to provide a composition for alleviating skin damage due to hypoxia, which contains one or more selected from the group consisting of a Saccharomyces cerevisiae strain; its lysate; its culture; and an extract of the strain, lysate, or culture as an active ingredient.

In another aspect, an object of the present invention is to provide a newly discovered Saccharomyces cerevisiae strain.

An aspect of the present invention provides a composition for alleviating skin damage due to hypoxia, which contains one or more selected from the group consisting of a Saccharomyces cerevisiae strain; its lysate; its culture; and an extract of the strain, lysate, or culture as an active ingredient.

Another aspect of the present invention provides a Saccharomyces cerevisiae GT23 strain with accession number KCCM13221P.

### BRIEF DESCRIPTION OF THE DRAWINGS

FIG. 1 is a graph illustrating the results of comparing the expression level of HIF1A, a hypoxia biomarker, before and after treatment of keratinocytes with CoCl₂ according to Example of the present invention;

FIG. 2 is a graph illustrating the results of comparing the expression level of MMP1 when keratinocytes in a hypoxic state prepared according to Example of the present invention are treated with an extract of a Saccharomyces cerevisiae GT23 strain of the present invention.

FIG. 3 is a graph illustrating the results of comparing the expression level of COX2 when keratinocytes in a hypoxic state prepared according to Example of the present invention are treated with an extract of a Saccharomyces cerevisiae GT23 strain of the present invention.

### DETAILED DESCRIPTION OF THE PREFERRED EMBODIMENTS

Hereinafter, the present invention will be described in detail.

In an aspect, the present invention provides a composition for alleviating skin damage due to hypoxia, which contains one or more selected from the group consisting of a Saccharomyces cerevisiae strain; its lysate; its culture; and an extract of the strain, lysate, or culture as an active ingredient.

The Saccharomyces cerevisiae strain according to an aspect of the present invention may be a Saccharomyces cerevisiae GT23 strain with the accession number KCCM13221P, and may specifically have DNA encoding 18S rRNA represented by SEQ ID NO: 1. The SEQ ID NO: 1 refers to the DNA sequence shown in Table 1 below.

TABLE 1
SEQ ID No: 1(5′→3′)

GCATTTATACAGTGAAACTGCGAATGGCTCATTAAATCAGTTATCGTTTA
TTTGATAGTTCCTTTACTACATGGTATAACTGTGGTAATTCTAGAGCTAA
TACATGCTTAAAATCTCGACCCTTTGGAAGAGATGTATTTATTAGATAAA
AAATCAATGTCTTCGGACTCTTTGATGATTCATAATAACTTTTCGAATCG
CATGGCCTTGTGCTGGCGATGGTTCATTCAAATTTCTGCCCTATCAACTT
TCGATGGTAGGATAGTGGCCTACCATGGTTTCAACGGGTAACGGGGAATA
AGGGTTCGATTCCGGAGAGGGAGCCTGAGAAACGGCTACCACATCCAAGG
AAGGCAGCAGGCGCGCAAATTACCCAATCCTAATTCAGGGAGGTAGTGAC
AATAAATAACGATACAGGGCCCATTCGGGTCTTGTAATTGGAATGAGTAC
AATGTAAATACCTTAACGAGGAACAATTGGAGGGCAAGTCTGGTGCCAGC
AGCCGCGGTAATTCCAGCTCCAATAGCGTATATTAAAGTTGTTGCAGTTA
AAAAGCTCGTAGTTGAACTTTGGGCCCGGTTGGCCGGTCCGATTTTTTCG
TGTACTGGATTTCCAACGGGGCCTTTCCTTCTGGCTAACCTTGAGTCCTT
GTGGCTCTTGGCGAACCGGGACTTTTACTTTGAAAAAATTAGAGTGTTCA
AAGCAGGCGTATTGCTCGAATATATTAGCATGGAATAATAGAATAGGACG
TTTGGTTCTATTTTGTTGGTTTCTAGGACCATCGTAATGATTAATAGGGA
CGGTCGGGGGCATCAGTATTCAATTGTCAGAGGTGAAATTCTTGGATTTA
TTGAAGACTAACTACTGCGAAAGCATTTGCCAAGGACGTTTTCATTAATC
AAGAACGAAAGTTAGGGGATCGAAGATGATCAGATACCGTCGTAGTCTTA
ACCATAAACTATGCCGACTAGGGATCGGGTGGTGTTTTTTTAATGACCCA
CTCGGCACCTTACGAGAAATCAAAGTCTTTGGGTTCTGGGGGGAGTATGG
TCGCAAGGCTGAAACTTAAAGGAATTGACGGAAGGGCACCACCAGGAGTG
GAGCCTGCGGCTTAATTTGACTCAACACGGGGAAACTCACCAGGTCCAGA
CACAATAAGGATTGACAGATTGAGAGCTCTTTCTTGATTTTGTGGGTGGT
GGTGCATGGCCGTTCTTAGTTGGTGGAGTGATTTGTCTGCTTAATTGCGA
TAACGAACGAGACCTTAACCTACTAAATAGTGGTGCTAGCATTTGCTGGT
TATCCACTTCTTAGAGGGACTATCGGTTTCAAGCCGATGGAAGTTTGAGG
CAATAACAGGTCTGTGATGCCCTTAGACGTTCTGGGCCGCACGCGCGCTA
CACTGACGGAGCCAGCGAGTCTAACCTTGGCCGAGAGGTCTTGGTAATCT
TGTGAAACTCCGTCGTGCTGGGGATAGAGCATTGTAATTATTGCTCTTCA
ACGAGGAATTCCTAGTAAGCGCAAGTCATCAGCTTGCGTTGATTACGTCC
CTGCCCTTTGTACACACCGCCCGTCGCTAGTACCGATTGAATGGCTTAGT
GAGGCCTCAGGATCTGCTTAGAGAAGGGGGCAACTCCATCTCAGAGCGGA
GAATT

The composition according to an aspect of the present invention may contain one or more selected from the group consisting of a Saccharomyces cerevisiae strain; its lysate; its culture; and an extract of the strain, lysate, or culture as an active ingredient.

The Saccharomyces cerevisiae strain according to an aspect of the present invention may be prepared and used in the form of a strain extract by culturing the strain, centrifuging the culture, removing the culture, and disrupting the strain.

The Saccharomyces cerevisiae strain according to an aspect of the present invention may be derived from green tea flowers.

The Saccharomyces cerevisiae strain according to an aspect of the present invention may have DNA encoding 18S rRNA represented by the sequence of SEQ ID NO: 1.

The lysate of the strain according to an aspect of the present invention may be a product obtained by disrupting the strain itself using chemical or physical force. The culture of the strain may be a substance containing some or all of the substances contained in the medium in which the strain was cultured, including the culture solution, regardless of the form of the culture, and may specifically be a substance containing metabolites or secretions resulting from strain culturing or a lysate thereof, and the strain itself may also be contained in the culture.

The culture of the strain according to an aspect of the present invention may include one obtained by culturing the strain in a culture medium containing yeast extract, peptone, or dextrose.

The extract of the strain according to an aspect of the present invention may be a product obtained by extracting the strain itself, a lysate of the strain, a culture of the strain, or a mixture thereof, regardless of the extraction method, extraction solvent, extracted component, or form of the extract, and may be a substance that can be obtained through processing or treatment by another method after extraction.

The composition according to an aspect of the present invention may contain one or more selected from the group consisting of a Saccharomyces cerevisiae strain; its lysate; its culture; and an extract of the strain, lysate, or culture at 0.001% to 30% by weight based on the total weight of the composition. Specifically, one or more selected from the group consisting of a Saccharomyces cerevisiae strain; its lysate; its culture; and an extract of the strain, lysate, or culture may be contained at 0.001% by weight or more, 0.01% by weight or more, 0.1% by weight or more, 0.5% by weight or more, 1% by weight or more, 1.5% by weight or more, or 2% by weight or more and 30% by weight or less, 25% by weight or less, 20% weight or less, 15% weight or less, 10% weight or less, or 5% weight or less based on the total weight of the composition.

Skin damage due to hypoxia according to an aspect of the present invention refers to skin damage of which the source of stimulus is a hypoxic state or a hypoxic environment. The skin damage is a broad concept that includes deterioration or weakening of skin function, specifically, skin damage due to hypoxia may include acceleration of skin aging, reduction of skin moisturizing power, or formation of skin wrinkles. Therefore, skin exposed to a hypoxic environment for a long period of time or repeatedly may undergo dryness, accelerated aging, and increased wrinkling.

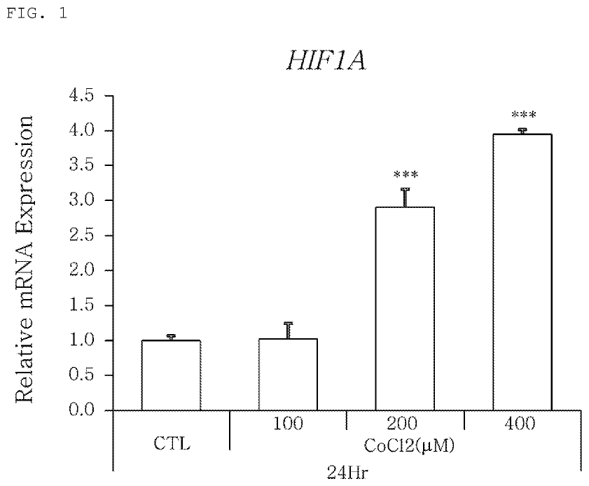

The skin damaged by hypoxia according to an aspect of the present invention may be skin having increased expression of one or more genes selected from the group consisting of hypoxia-inducible factor 1-alpha (HIF1A), matrix metalloproteinase-1 (MMP1), and cytochrome c oxidase subunit 2 (COX2). According to an example of the present invention, the expression levels of H1F1A, MMP1, and COX2 were increased in skin exposed to a hypoxic environment compared with those before exposure (Experimental Example, FIG. 1).

The alleviation of skin damage due to hypoxia of the present invention may be anti-aging or wrinkle improvement.

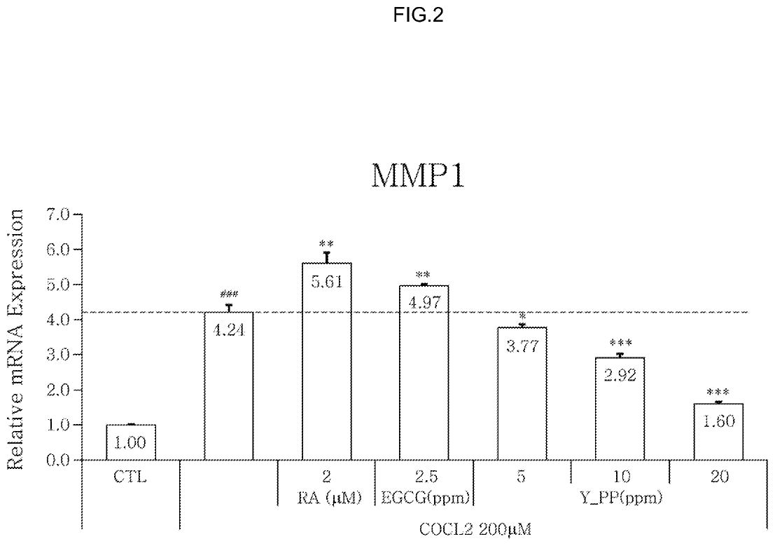

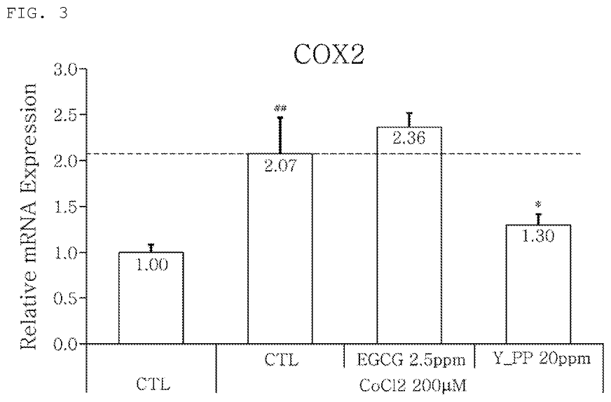

The composition according to an aspect of the present invention can reduce the expression level of one or more genes selected from the group consisting of MMP1 and COX2. Specifically, the composition according to an aspect of the present invention can reduce the expression level of one or more genes selected from the group consisting of MMP1 and COX2 by 5% or more, 6% or more, 8% or more, 10% or more, 12% or more, 14% or more, 16% or more, 18% or more, 20% or more, 22% or more, 24% or more, 26% or more, 28% or more, 30% or more, 32% or more, 34% or more, 36% or more, 38% or more, 40% or more, 42% or more, 44% or more, 46% or more, or 48% or more and 50% or less, 48% or less, 46% or less, 44% or less, 42% or less, 40% or less, 38% or less, 36% or less, 34% or less, 32% or less, 30% or less, 28% or less, 26% or less, 24% or less, 22% or less, 20% or less, 18% or less, 16% or less, 14% or less, 12% or less, 10% or less, 8% or less, or 6% or less compared with that before treatment with the composition. According to Example of the present invention, when skin damaged by hypoxia was treated with one or more selected from the group consisting of a Saccharomyces cerevisiae strain; its lysate; its culture; and an extract of the strain, lysate, or culture according to an aspect of the present invention, the expression level of MMP1 or COX2 was reduced compared with that before treatment (Experimental Example, FIGS. 2 and 3).

In an aspect of the present invention, the composition may be a pharmaceutical composition, cosmetic composition, or food composition.

The pharmaceutical composition according to an aspect of the present invention may be formulated as a preparation for oral or parenteral administration in a solid, semi-solid or liquid form by adding a commonly used inorganic or organic carrier to the composition used as an active ingredient.

Examples of the preparation for oral administration include tablets, pills, granules, soft and hard capsules, powder remedies, fine granules, powders, emulsions, syrups, and pellets. Examples of the preparation for parenteral administration include injections, drops, ointments, lotions, sprays, suspensions, emulsions, and suppositories. In order to formulate the active ingredient of the present invention, the active ingredient may be easily formulated by carrying out a conventional method, and surfactants, excipients, colorants, spices, preservatives, stabilizers, buffers, suspending agents, and other commonly used auxiliaries may be used appropriately.

The pharmaceutical composition according to the present invention is excellent in alleviating skin damage due to hypoxia, and can be usefully used in the treatment or prevention of skin diseases caused by skin damage due to hypoxia. Skin diseases caused by skin damage due to hypoxia include, but are not limited to, atopic dermatitis, xeroderma, psoriasis, ichthyosis, and acne.

The pharmaceutical composition according to an aspect of the present invention may be administered orally, parenterally, rectally, topically, transdermally, intravenously, intramuscularly, intraperitoneally, subcutaneously, or the like.

The dosage of the active ingredient may vary depending on the age, sex, and weight of the subject to be treated, the specific disease or pathological state to be treated, the severity of the disease or pathological state, the route of administration, and the judgment of the prescriber. The dosage determination based on these factors is within the level of those skilled in the art. A typical dosage is 0.001 mg/kg/day to 2000 mg/kg/day, specifically 0.5 mg/kg/day to 1500 mg/kg/day.

The cosmetic composition according to an aspect of the present invention may be provided in any formulation suitable for topical application. For example, the cosmetic composition may be provided in the formulation of a liquid, emulsion obtained by dispersing an oil phase in an aqueous phase, emulsion obtained by dispersing an aqueous phase in an oil phase, suspension, solid, gel, powder, paste, foam or aerosol composition. A composition in such a formulation may be prepared according to a conventional method in the art.

The cosmetic composition according to an aspect of the present invention may contain other ingredients in addition to the above-mentioned substances within the range in which the main effect is not impaired, specifically those that can enhance the main effect. The cosmetic composition according to an aspect of the present invention may contain a substance selected from the group consisting of vitamins, high molecular weight peptides, molecular polysaccharides, and sphingolipids. The cosmetic composition according to an aspect of the present invention may contain moisturizers, emollients, surfactants, ultraviolet absorbers, preservatives, disinfectants, antioxidants, pH adjusters, organic and inorganic pigments, fragrances, cooling agents, or antiperspirants. The amounts of the ingredients blended may be easily selected by those skilled in the art within a range in which the objects and effects of the present invention are not impaired, and the blended amount may be 0.001% to 5% by weight, specifically 0.01% to 3% by weight based on the total weight of the composition.

The pharmaceutical composition or cosmetic composition according to an aspect of the present invention may be a composition for external application to the skin. The pharmaceutical composition or cosmetic composition may be, for example, a cream or ointment.

The food composition according to an aspect of the present invention may be a health food composition, and may be a fermented food composition that requires fermentation, such as tea, dairy products, kimchi, and brewed foods. The formulation of the food composition is not particularly limited, but the food composition may be formulated into, for example, tablets, pills, soft and hard capsules, granules, health drinks, caramels, diet bars, and tea bags. In addition to the active ingredient, in the food composition in each formulation, ingredients commonly used in the field may be appropriately selected and blended by those skilled in the art depending on the formulation or purpose of use without difficulties, and a synergistic effect may be exhibited when the active ingredient is applied simultaneously with other raw materials. Determination of the dosage of the active ingredient is within the level of those skilled in the art, and the dosage may vary depending on various factors such as the age, health status, and complications of the subject to which the active ingredient is administered.

The food composition according to the present invention is excellent in alleviating skin damage due to hypoxia, and can be usefully used in the improvement or prevention of skin diseases caused by skin damage due to hypoxia. Skin diseases caused by skin damage due to hypoxia include, but are not limited to, atopic dermatitis, xeroderma, psoriasis, ichthyosis, and acne.

In another aspect, the present invention provides a Saccharomyces cerevisiae GT23 strain with accession number KCCM13221P.

The Saccharomyces cerevisiae GT23 strain according to an aspect of the present invention has a function of alleviating skin damage due to hypoxia. The skin damage due to hypoxia, skin damaged by hypoxia, alleviation of skin damage due to hypoxia, and the like are as described above.

The Saccharomyces cerevisiae GT23 strain according to an aspect of the present invention may be derived from green tea flowers.

From another perspective, the present invention may relate to a method for alleviating skin damage due to hypoxia, which includes applying or administering one or more selected from the group consisting of a Saccharomyces cerevisiae strain; its lysate; its culture; and an extract of the strain, lysate, or culture to a subject in need of alleviation of skin damage due to hypoxia.

From still another perspective, the present invention may relate to a method for improving, preventing, or treating skin diseases caused by skin damage due to hypoxia, which includes applying or administering one or more selected from the group consisting of a Saccharomyces cerevisiae strain; its lysate; its culture; and an extract of the strain, lysate, or culture to a subject who has a skin disease caused by skin damage due to hypoxia, specifically atopic dermatitis, xeroderma, psoriasis, ichthyosis or acne and needs improvement, prevention, or treatment of the skin damage or skin disease.

From still another perspective, the present invention may relate to a use of one or more selected from the group consisting of a Saccharomyces cerevisiae strain; its lysate; its culture; and an extract of the strain, lysate, or culture for preparation of a pharmaceutical composition for alleviating skin damage due to hypoxia.

From still another perspective, the present invention may relate to a use of one or more selected from the group consisting of a Saccharomyces cerevisiae strain; its lysate; its culture; and an extract of the strain, lysate, or culture for preparation of a cosmetic composition for alleviating skin damage due to hypoxia.

From still another perspective, the present invention may relate to a use of one or more selected from the group consisting of a Saccharomyces cerevisiae strain; its lysate; its culture; and an extract of the strain, lysate, or culture for preparation of a food composition for alleviating skin damage due to hypoxia.

The present invention may provide the following embodiments as Example.

A first embodiment may provide a method for alleviating skin damage due to hypoxia, comprising administering an effective amount of a composition containing one or more selected from the group consisting of a Saccharomyces cerevisiae strain; a lysate of the strain; a culture of the strain; and an extract of the strain, lysate, or culture to a subject in need of alleviation of skin damage due to hypoxia.

A second embodiment may provide the method according to the first embodiment, in which the strain is Saccharomyces cerevisiae GT23 with accession number KCCM13221P.

A third embodiment may provide the method according to the first or second embodiment, in which the Saccharomyces cerevisiae strain is derived from a green tea flower.

A fourth embodiment may provide the method according to one or more of the first to third embodiments, in which the strain has DNA encoding 18S rRNA represented by a sequence of SEQ ID NO: 1.

A fifth embodiment may provide the method according to one or more of the first to fourth embodiments, in which the active ingredient is contained at 0.001% to 30% by weight based on a total weight of the composition.

A sixth embodiment may provide the method according to one or more of the first to fifth embodiments, in which the skin damaged by hypoxia is skin having increased expression of one or more genes selected from the group consisting of hypoxia-inducible factor 1-alpha (HIF1A), matrix metalloproteinase-1 (MMP1), and cytochrome c oxidase subunit 2 (COX2).

A seventh embodiment may provide the method according to one or more of the first to sixth embodiments, in which the alleviation of skin damage due to hypoxia is anti-aging or wrinkle improvement.

An eighth embodiment may provide the method according to one or more of the first to seventh embodiments, in which the composition reduces an expression level of one or more genes selected from the group consisting of MMP1 and COX2.

A ninth embodiment may provide the method according to one or more of the first to eighth embodiments, in which the composition is a pharmaceutical composition.

A tenth embodiment may provide the method according to one or more of the first to eighth embodiments, in which the composition is a cosmetic composition.

An eleventh embodiment may provide the method according to one or more of the first to eighth embodiments, in which the composition is a food composition.

A twelfth embodiment may provide a Saccharomyces cerevisiae GT23 strain with accession number KCCM13221P.

A thirteenth embodiment may provide the strain according to twelfth embodiment, which has a function of alleviating skin damage due to hypoxia.

A fourteenth embodiment may provide the strain according to twelfth or thirteenth embodiment, which is derived from a green tea flower.

Hereinafter, the configuration and effects of the present invention will be described in more detail through Examples and Experimental Examples. However, the following Examples, Preparation Examples, and Experimental Examples are provided only for illustrative purposes to aid understanding of the present invention, and the gist and scope of the present invention are not limited thereto.

### [Example 1] Isolation and Identification of Strain

The Saccharomyces cerevisiae strain was isolated from green tea flowers at OSULLOC Tea Garden in Jeju, and the isolation method was as follows.

First, 10 g of collected green tea flowers were placed in 50 ml of PBS buffer, and stirring was performed for 2 hours. Thereafter, impurities were removed using filter paper, and the filtered PBS was diluted with YPD (yeast extract peptone dextrose) culture medium. Then, the diluted PBS was plated on YPD agar (solid medium) and cultured at 28° C.

Thereafter, in order to genetically identify the isolated strain, 18s rRNA sequencing was performed using the ITS1 primer 5′-TCCGTAGCTGAACCTGCGG-3′ (SEQ ID NO: 2). The result of sequencing the ITS1 partial sequence was identified through homology comparison with known sequences in NCBI blast, and as a result, the isolated strain was confirmed to have high homology with Saccharomyces cerevisiae, named Saccharomyces cerevisiae GT23, deposited at the Korean Culture Center of Microorganisms (KCCM), a depository authority, at Yurim B/D, 45, Hongjenae-2ga-gil, Seodaemun-gu, Seoul 03641, Republic of Korea, on Jul. 29, 2022 under the Budapest Treaty on the International Recognition of the Deposit of Microorganisms for the Purposes of Patent Procedure, and given the accession number KCCM13221P.

With regard to the deposit of accession number KCCM13221P, Applicant agrees that the instant invention will be irrevocably and without restriction released to the public upon issuance of a patent, that the above-identified deposited material be maintained for a period of 30 years or 5 years after the last request for the deposit, or for the effective life of any patent which issues on the above-identified application, whichever is longer, that if the deposit (s) become non-viable, it/they will be replaced, and also assure that access to the deposit (s) will be available during pendency of the above-identified application to one determined by the Director to be entitled thereto under 37 C.F.R. § 1.14 and 35 U.S.C. § 122.

### [Example 2] Preparation of Extract of Saccharomyces cerevisiae GT23 Strain

In order to prepare a microbial culture solution, the Saccharomyces cerevisiae GT23 strain of Example 1 was inoculated into YPD medium (yeast extract 10 g/L, peptone 20 g/L, dextrose 20 g/L) and cultured at 28° C. for 2 days. After that, the cultured microorganism was recovered by centrifugation (8000 rpm), the culture was removed, and the strain was washed using distilled water. Next, the obtained strain was disrupted to prepare a strain extract (Y_PP).

### [Experimental Example] Verification of Effect on Alleviation of Skin Damage Due to Hypoxia

First, keratinocytes (HEKn) (onza, Switzerland) were treated with 200 μM CoCl₂ to induce a hypoxic environment. After that, the expression level of hypoxia-inducible factor 1-alpha (HIF1A) that was known to be expressed under hypoxia was compared with that before the treatment with CoCl₂ (CTL in FIG. 1) to confirm that the expression level of HIF1A increased compared with that before the treatment with CoCl₂ (FIG. 1), and thus skin (keratinocytes) damaged by hypoxia (hereinafter referred to as hypoxic keratinocytes) was prepared.

Thereafter, in order to verify the effect of the strain isolated in Example 1 on the alleviation of skin damage due to hypoxia, the hypoxic keratinocytes were treated with the extract prepared in Example 2 at various concentrations and then cultured for 18 hours. The cultured keratinocytes were collected, RNA was extracted, CDNA was synthesized from the extracted RNA, and qPCR (quantitative real-time PCR) was performed. Specifically, 10 μL of universal master mix (Taqman 2X Universal PCR mix, Applied Biosystems, Cat. No. 4304437) and 1 μL of TaqMan probe were added, 5 μL of CDNA was added to obtain a total volume of 20 μL, and PCR was performed using the Applied biosystem 7500 fast real time PCR system and the provided standard protocol. The expression of MMP1, an aging-related gene, and COX2, an inflammation-related gene, was examined through qPCR, and the results are illustrated in FIGS. 2 and 3. At this time, EGCG was used as Comparative Example. In FIG. 2, the groups on the x-axis are as follows in order from the left: CTL denotes the group not treated with CoCl₂, and among the groups treated with CoCl₂, the leftmost group denotes the group treated with CoCl₂ and no other substances, RA denotes the group treated with CoCl₂ and 2 μM retinoic acid, EGCG denotes the group treated with CoCl₂ and 2.5 UM EGCG, and Y_PP denotes the groups treated with CoCl₂ and the extract prepared in Example 2 at 5, 10, and 20 ppm, respectively. In FIG. 3, the groups on the x-axis are as follows in order from the left: CTL denotes the group not treated with CoCl₂, and among the groups treated with CoCl₂, the leftmost CTL denotes the group treated with CoCl₂ and no other substances, EGCG denotes the group treated with CoCl₂ and EGCG at 2.5 ppm, and Y_PP denotes the group treated with CoCl₂ and the extract prepared in Example 2 at 20 ppm.

As can be seen from FIGS. 2 and 3, the expression levels of MMP1 and COX2 have increased in skin exposed to a hypoxic environment, and it has been found that skin in a hypoxic state is damaged in the form of accelerated aging or increased wrinkling.

As illustrated in FIG. 2, it has been confirmed that EGCG known as a representative MMP1 inhibitor cannot inhibit MMP1 overexpressed by hypoxia but the extract of Example 2 obtained from the strain isolated in Example 1 is effective in inhibiting overexpression of MMP1 due to hypoxia.

As illustrated in FIG. 3, in the case of COX2, it has been confirmed that EGCG cannot inhibit expression of COX2 overexpressed by hypoxia but the extract of Example 2 obtained from the strain isolated in Example 1 is effective in inhibiting overexpression of COX2 due to hypoxia, similar to the case of MMP1.

Through this, it has been found that the Saccharomyces cerevisiae strain; its lysate; its culture; and an extract of the strain, lysate, or culture according to an aspect of the present invention has an excellent effect in alleviating skin damage due to hypoxia.

Formulation Examples of the composition according to an aspect of the present specification will be described below, but the composition can be applied as various other formulations, and this is not intended to limit the present invention but is only intended to provide a detailed description.

### [Formulation Example 1] Softening Lotion

A softening lotion was prepared by mixing 0.01% by weight of the Saccharomyces cerevisiae extract of Example 2, 3% by weight of glycerin, 2% by weight of butylene glycol, 2% by weight of propylene glycol, 0.1% by weight of carboxyvinyl polymer, 10% by weight of ethanol, 0.1% by weight of triethanolamine, the remaining amount of preservative, a trace amount of coloring agent, a trace amount of fragrance, and a trace amount of purified water according to a conventional softening lotion preparation method.

### [Formulation Example 2] Nutritional Lotion

A nutritional lotion was prepared by mixing 0.01% by weight of Saccharomyces cerevisiae extract of Example 2, 4% by weight of beeswax, 1.5% by weight of polysorbate 60, 0.5% by weight of sorbitan sesquioleate, 5% by weight of liquid paraffin, 5% by weight of squalane, 5% by weight of caprylic/capric triglyceride, 3% by weight of glycerin, 3% by weight of butylene glycol, 3% by weight of propylene glycol, 0.1% by weight of carboxyvinyl polymer, 0.2% by weight of triethanolamine, the remaining amount of preservative, a trace amount of coloring agent, a trace amount of fragrance, and a trace amount of purified water according to a conventional nutritional lotion preparation method.

### [Formulation Example 3] Nourishing Cream

A nourishing cream was prepared by mixing 0.01% by weight of the Saccharomyces cerevisiae extract of Example 2, 10% by weight of beeswax, 1.5% by weight of polysorbate 60, 0.5% by weight of sorbitan sesquioleate, 10% by weight of liquid paraffin, 5% by weight of squalane, 5% by weight of caprylic/capric triglyceride, 5% by weight of glycerin, 3% by weight of butylene glycol, 3% by weight of propylene glycol, 0.2% by weight of triethanolamine, the remaining amount of preservative, a trace amount of coloring agent, a trace amount of fragrance, and a trace amount of purified water according to a conventional nourishing cream preparation method.

### [Formulation Example 4] Sheet Mask

A sheet mask was prepared by mixing 0.01% by weight of the Saccharomyces cerevisiae extract of Example 2, 13% by weight of polyvinyl alcohol, 0.2% by weight of sodium carboxymethyl cellulose, 0.1% by weight of allantoin, 5% by weight of ethanol, 0.3% by weight of nonylphenyl ether, the remaining amount of preservative, a trace amount of coloring agent, a trace amount of fragrance, and a trace amount of purified water according to a conventional pack preparation method.

### [Formulation Example 5] Preparation of Medication (Patch) for Topical Administration

A medication (patch) for topical administration was prepared by a conventional method according to the composition shown in Table 3 below.

TABLE 3

| | Content |
|---|---|
| Raw material name | (% by weight) |
| Saccharomyces cerevisiae extract of | 2.0 |
| Example 2 | |
| Beta-1,3-glucan | 3.0 |
| Diethylamine | 0.7 |
| Sodium sulfite | 0.1 |
| Polyoxyethylene lauryl ether (E.O = 9) | 1.0 |
| Polyhydroxyethylene cetyl/stearyl ether | 1.0 |
| (Cetomacrogol 1000) | |
| Viscous paraffin oil | 2.5 |
| Caprylic acid ester/capric acid ester | 2.5 |
| (Cetiol LC) | |
| Polyethylene glycol 400 | 3.0 |
| Polyacrylic acid (Carbopol 934P) | 1.0 |
| Purified water | Remaining amount |
| Sum | 100 |

### [Formulation Example 6] Preparation of Powder

A powder was prepared by mixing 2 g of the Saccharomyces cerevisiae extract of Example 2 and 1 g of lactose and then filling the mixture into an airtight bag.

### [Formulation Example 7] Preparation of Tablet

A tablet was prepared by mixing 100 mg of the Saccharomyces cerevisiae extract of Example 2, 100 mg of corn starch, 100 mg of lactose, and 2 mg of magnesium stearate and then tableting the mixture according to a conventional tablet preparation method.

### [Formulation Example 8] Preparation of Capsule

A capsule was prepared by mixing 100 mg of the Saccharomyces cerevisiae extract of Example 2, 100 mg of corn starch, 100 mg of lactose, and 2 mg of magnesium stearate and then filling the mixture into a gelatin capsule according to a conventional capsule preparation method.

### [Formulation Example 9] Preparation of Pill

After 1 g of the Saccharomyces cerevisiae extract of Example 2, 1.5 g of lactose, 1 g of glycerin, and 0.5 g of xylitol were mixed, the mixture was prepared into pills to be 4 g per pill according to a conventional method.

### [Formulation Example 10] Preparation of Granule

After 150 g of the Saccharomyces cerevisiae extract of Example 2, 50 mg of soybean extract, 200 mg of glucose, and 600 mg of starch were mixed, 100 mg of 30% ethanol was added, drying was performed at 60° ° C. to form granules, and then the granules were filled into bags.

### [Formulation Example 11] Health Drink

After 50 mg of the Saccharomyces cerevisiae extract of Example 2, 10 g of glucose, 0.6 g of citric acid, and 25 g of liquid oligosaccharide were mixed, 300 ml of purified water was added, and the mixture was filled into each bottle by 200 ml. After being filled into bottles, the mixture was sterilized at 130° C. for 4 to 5 seconds to prepare a beverage.

### [Formulation Example 12] Caramel Formulation

Caramel was formed by mixing 50 mg of the Saccharomyces cerevisiae extract of Example 2, 1.8 g of corn syrup, 0.5 g of skim milk, 0.5 g of soy lecithin, 0.6 g of butter, 0.4 g of hydrogenated vegetable oil, 1.4 g of sugar, 0.58 g of margarine, and 20 mg of table salt.

As above, specific parts of the present invention have been described in detail, and it will be clear to those skilled in the art that the specific description is merely preferred embodiments and do not limit the scope of the present invention. Accordingly, the actual scope of the present invention will be defined by the appended claims and their equivalents.

### [Microorganism Deposit]

Name of depository authority: Korea Culture Center of Microorganisms (KCCM)
Accession number: KCCM13221P
Accession date: Jul. 29, 2022

The composition, which contains one or more selected from the group consisting of a Saccharomyces cerevisiae strain; its lysate; its culture; and an extract of the strain, lysate, or culture as an active ingredient, according to an aspect of the present invention is effective in inhibiting the expression of MMP1 and COX2 overexpressed in skin in a hypoxic environment, and thus has an excellent effect in improving skin damaged by exposure to a hypoxic environment for a long period of time or repeatedly or alleviating skin damage due to hypoxia.

While the present invention has been described with respect to the specific embodiments, it will be apparent to those skilled in the art that various changes and modifications may be made without departing from the spirit and scope of the invention as defined in the following claims.

What is claimed is:

1. A method for alleviating skin damage due to hypoxia, comprising administering an effective amount of a composition containing one or more selected from the group consisting of a Saccharomyces cerevisiae strain; a lysate of the strain; a culture of the strain; and an extract of the strain, lysate, or culture to a subject in need of alleviation of skin damage due to hypoxia.

2. The method according to claim 1,
wherein the strain is Saccharomyces cerevisiae GT23 with accession number KCCM13221P.

3. The method according to claim 1,
wherein the Saccharomyces cerevisiae strain is derived from a green tea flower.

4. The method according to claim 2,
wherein the strain has DNA encoding 18S rRNA represented by a sequence of SEQ ID NO: 1.

5. The method according to claim 1,
wherein the active ingredient is contained at 0.001% to 30% by weight based on a total weight of the composition.

6. The method according to claim 1,
wherein the skin damaged by hypoxia is skin having increased expression of one or more genes selected from the group consisting of hypoxia-inducible factor 1-alpha (HIF1A), matrix metalloproteinase-1 (MMP1), and cytochrome c oxidase subunit 2 (COX2).

7. The method according to claim 1,
wherein the alleviation of skin damage due to hypoxia is anti-aging or wrinkle improvement.

8. The method according to claim 1,
wherein the composition reduces an expression level of one or more genes selected from the group consisting of MMP1 and COX2.

9. The method according to claim 1, wherein the composition is a pharmaceutical composition.

10. The method according to claim 1, wherein the composition is a cosmetic composition.

11. The method according to claim 1, wherein the composition is a food composition.

12. A Saccharomyces cerevisiae GT23 strain with accession number KCCM13221P.

13. The strain according to claim 12, which has a function of alleviating skin damage due to hypoxia.

14. The strain according to claim 12, which is derived from a green tea flower.


SEQUENCE LISTING (2 sequences)

SEQ ID NO: 1 - DNA, 1655 bp, Saccharomyces cerevisiae GT23
  source 1..1655: mol_type = genomic DNA
gcatttatacagtgaaactgcgaatggctcattaaatcagttatcgtttatttgatagttcctttactacatggtataactgtggtaattctagagctaatacatgcttaaaatctcgaccctttggaagagatgtatttattagataaaaaatcaatgtcttcggactctttgatgattcataataacttttcgaatcgcatggccttgtgctggcgatggttcattcaaatttctgccctatcaactttcgatggtaggatagtggcctaccatggtttcaacgggtaacggggaataagggttcgattccggagagggagcctgagaaacggctaccacatccaaggaaggcagcaggcgcgcaaattacccaatcctaattcagggaggtagtgacaataaataacgatacagggcccattcgggtcttgtaattggaatgagtacaatgtaaataccttaacgaggaacaattggagggcaagtctggtgccagcagccgcggtaattccagctccaatagcgtatattaaagttgttgcagttaaaaagctcgtagttgaactttgggcccggttggccggtccgattttttcgtgtactggatttccaacggggcctttccttctggctaaccttgagtccttgtggctcttggcgaaccgggacttttactttgaaaaaattagagtgttcaaagcaggcgtattgctcgaatatattagcatggaataatagaataggacgtttggttctattttgttggtttctaggaccatcgtaatgattaatagggacggtcgggggcatcagtattcaattgtcagaggtgaaattcttggatttattgaagactaactactgcgaaagcatttgccaaggacgttttcattaatcaagaacgaaagttaggggatcgaagatgatcagataccgtcgtagtcttaaccataaactatgccgactagggatcgggtggtgtttttttaatgacccactcggcaccttacgagaaatcaaagtctttgggttctggggggagtatggtcgcaaggctgaaacttaaaggaattgacggaagggcaccaccaggagtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctactaaatagtggtgctagcatttgctggttatccacttcttagagggactatcggtttcaagccgatggaagtttgaggcaataacaggtctgtgatgcccttagacgttctgggccgcacgcgcgctacactgacggagccagcgagtctaaccttggccgagaggtcttggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctagtaccgattgaatggcttagtgaggcctcaggatctgcttagagaagggggcaactccatctcagagcggagaatt

SEQ ID NO: 2 - DNA, 19 bp, synthetic construct
  source 1..19: mol_type = other DNA
tccgtagctgaacctgcgg